(19) United States
(12) Patent Application Publication
(10) Pub. No.: US 2024/0108815 A1
(43) Pub. Date: Apr. 4, 2024

(54) Autoinjector for Automatic Injection of a Product Into an Injection Site

(71) Applicant: Becton Dickinson France, Le Pont-de-Claix (FR)

(72) Inventor: Michael Fiard, Eybens (FR)

(21) Appl. No.: 18/375,668

(22) Filed: Oct. 2, 2023

(30) Foreign Application Priority Data
Oct. 3, 2022 (EP) 22306466.8

(51) Int. Cl. A61M 5/32; A61M 5/20

(52) U.S. Cl. CPC A61M 5/3232; A61M 5/20; A61M 5/3202

(57) ABSTRACT

This autoinjector includes a housing extending along longitudinal axis A and having an opened distal end, said housing being configured to receive a medical container having a barrel defining a reservoir for containing a medical product, said barrel having a distal end provided with an injection needle. A needle cover is coupled to and axially movable with respect to said housing between a first extended position, a retracted position, and a second extended position. A cap is removably attached to the opened distal end of the housing. A retainer is coupled to the housing for blocking a proximal movement of the needle cover from the first extended position to the retracted position as long as the cap is attached to the housing. The retainer includes a distal abutment surface which is radially movable between a blocking position in which said distal abutment surface abuts against a proximal abutment surface of the needle cover, and a release position in which said distal abutment surface is radially shifted away from the proximal abutment surface of the needle cover. The cap includes a locking tab radially abutting against the retainer for maintaining the distal abutment surface of the retainer in the blocking position.

### CROSS-REFERENCE TO RELATED APPLICATION

This application claims priority to European Patent Application No. 22306466.8 filed Oct. 3, 2022, the disclosure of which is hereby incorporated by reference in its entirety.

### BACKGROUND OF THE INVENTION

### Field of the Invention

The present invention relates to an autoinjector.

### Description of Related Art

In this application, the distal end of a component or of a device is to be understood as meaning the end furthest from the user's hand and the proximal end is to be understood as meaning the end closest to the user's hand. Likewise, in this application, the “distal direction” is to be understood as meaning the direction away from the user's hand, and the “proximal direction” is to be understood as meaning the direction toward the user's hand.

Also, by outer it is meant centrifugally oriented with regard to a central longitudinal axis, and by inner it is meant centripetally with regard to said central longitudinal axis.

Automatic injection devices are designed for automatic injection of a medical product into an injection site. Autoinjectors usually comprise a housing for receiving a medical container having a barrel defining a reservoir for containing the medical product, the barrel having a distal end provided with an injection needle and an opened proximal end receiving a plunger rod for pushing a stopper. The opened proximal end is usually provided with a flange.

Autoinjectors also include a needle cover moving from an extended to a retracted position to shield or unveil the needle and an injection mechanism for automatically injecting the medical into an injection site. The injection mechanism is usually triggered, directly or indirectly, by the needle cover when the needle cover moves to the retracted position. It is important to prevent untimely triggering or pre-activation of the autoinjector, for example during transport or storage. The needle covers are usually held or urged back in their extended position by a safety spring.

However, to assess the robustness of the autoinjectors, autoinjectors are subjected to drop tests as required in ISO11608. These drop tests usually consist in dropping the autoinjectors at least once from a height of 1m onto a horizontal floor. There are three drop directions : a drop ‘cap upward’ (the proximal end of the autoinjector hits the floor first), a drop ‘cap downward’ (the distal end of the autoinjector hits the floor first), and a drop with the autoinjector being horizontal.

During a drop ‘cap upward’, the kinetic energy released when the autoinjector hits the ground may cause the needle cover to suddenly move towards the retracted position against the action of the safety spring. There is thus a risk that the autoinjector is inadvertently activated.

There is accordingly a need for an autoinjector that is able to avoid activation of the injection mechanism during a drop test.

### SUMMARY OF THE INVENTION

An aspect of the invention is an autoinjector, for automatic injection of a product into an injection site, said autoinjector including:

a housing extending along longitudinal axis A and having an opened distal end, said housing being configured to receive a medical container having a barrel defining a reservoir for containing a medical product, said barrel having a distal end provided with an injection needle,

a needle cover coupled to and axially movable with respect to said housing between a first extended position, in which the needle cover at least partially shields the injection needle, a retracted position, in which the needle cover moves proximally with respect to the housing to trigger injection of the medical product into an injection site, and a second extended position in which the needle cover moves back in the distal direction to shield the injection needle,

a cap removably attached to the opened distal end of the housing,

a retainer coupled to the housing for blocking a proximal movement of the needle cover from the first extended position to the retracted position as long as the cap is attached to the housing, wherein the retainer includes

a distal abutment surface which is radially movable between a blocking position in which said distal abutment surface abuts against a proximal abutment surface of the needle cover, and a release position in which said distal abutment surface is radially shifted away from the proximal abutment surface of the needle cover to allow movement of the needle cover in the proximal direction, and wherein the cap includes

a locking tab radially abutting against the retainer for maintaining the distal abutment surface of the retainer in the blocking position.

As a result, the autoinjector of the invention is prevented from being activated or pre-activated. As long as the cap is attached to the housing, the retainer cannot move to the release position. Thus, the needle cover cannot move to the retracted position. There is thus no risk that the injection mechanism is triggered. The autoinjector of the invention can thus successfully pass drop tests. Inadvertent activation of the injection mechanism is avoided.

The proximal abutment surface may be arranged on an outer lateral wall of the needle cover. The locking tab is configured for radially abutting against the retainer so as to lock the retainer in the blocking position.

The autoinjector of the invention may further include some or all of the features below.

In an embodiment, the locking tab has an outer abutment surface abutting against an inner lateral wall of the housing. This helps maintain the locking tab in abutment against the retainer such that the retainer reliably blocks the needle cover.

In an embodiment, the retainer defines an accommodation room for receiving a free proximal end of the locking tab between an outer lateral surface of the retainer and an inner lateral wall of the housing.

Preferably, the locking tab has a proximally tapering width.

In an embodiment, an outer lateral surface of the free proximal end of the locking tab is separated from the inner lateral wall of the housing by a radial gap. The radial dimension of this gap may be greater than the radial dimension (thickness) of the proximal end of the locking tab.

In an embodiment, the locking tab has an axial rib distally extending from the free proximal end of the locking tab and said axial rib is fixed to an external wall of the retainer.

In an embodiment, the locking tab has an axial rib distally extending from the free proximal end of the locking tab and said axial rib has a distal end which is fixed to a connecting member that connects a cap external wall to a remover of the cap.

In an embodiment, the locking tab has a stiffener arranged at an outer side of the free proximal end, the stiffener proximally protruding from a distal edge of the cap.

These features provide support to the locking tab and distribute the efforts received by the locking tab, thus avoiding deformation of the locking tab. As a result, the retainer is reliably maintained by the locking tab in the blocking position.

In an embodiment, the stiffener of the locking tab includes an inclined surface for easing insertion of the locking tab inside the housing.

In an embodiment, the retainer includes a flexible leg, said flexible leg having a fixed end secured to the housing and a free end defining the distal abutment surface of the retainer. The flexible leg permits movement of the retainer to the release position under the force exerted by the user or by the safety spring on the needle cover after removal of the cap.

In an embodiment, the free end of the flexible leg is distal to the fixed end. Therefore, the retainer acts as a ratchet. This provides a better resistance to any proximal movement of the needle cover during a drop test.

Possibly, the opened distal end of the housing is distal to the free end of the flexible leg.

Possibly, the free distal end is closer to the central longitudinal axis than the fixed end of the retainer, at least when the retainer is in the blocking position.

In an embodiment, the fixed end of the flexible leg includes an annular rib radially protruding from an inner lateral wall of the housing. This eases movement of the retainer from the blocking position to the release position after removal of the cap. This also helps define the accommodation room between the retainer and the housing for receiving the locking tab.

In an embodiment, the blocking position of the retainer is a rest position of the flexible leg. That is, the flexible leg is in its natural (not deformed) state in the blocking position. This makes the blocking action of the retainer more reliable.

In an alternative embodiment, the release position of the retainer is a rest position. That is, the flexible leg is in its natural (not deformed) state in the release position. The retainer thus has to be pressed against the needle cover to stay in the blocking position. However, this limits the risks that the retainer inadvertently blocks the needle cover after the injection process when the needle cover moves back in the distal direction towards the second extended position (safety position).

In both embodiments, movement of the distal abutment of the retainer from the blocking to the release position may be a radial outward movement.

In an embodiment, the retainer has a proximal abutment surface abutting against a distal abutment surface of the needle cover for blocking the needle cover in the distal direction. Thus, the needle cover is locked in the first extended position as long as the cap is attached to the housing. The distal and/or the proximal abutment surface of the retainer may be inclined with regard to the central longitudinal axis A so that the retainer moves to the release position under the pressure of the end user or the action of the safety spring. The distal and/or proximal abutment surface of the needle cover may also be inclined with regard to the longitudinal axis A.

In an embodiment, the autoinjector includes securing means for removably securing the cap to the housing in both axial and rotational directions. This avoids inadvertent disengagement of the locking tab and the retainer.

Preferably, the securing means include an orthoradial shoulder of the housing abutting against an opposite orthoradial edge of the cap. This prevents rotation of the cap relative to the housing when the cap is attached to the housing, so that the locking tab cannot rotationally disengage the retainer.

The orthoradial shoulder of the housing abutting against an opposite orthoradial edge of the cap also form keyed elements for allowing proper positioning of the cap relative to the housing. Thus, the locking tab of the cap reliably engages the retainer of the housing when the cap is being mounted onto the housing.

### BRIEF DESCRIPTION OF THE DRAWINGS

The terms Fig., Figs., Figure, and Figures are used interchangeably in the specification to refer to the corresponding figures in the drawings.

The invention and the advantages arising therefrom will clearly emerge from the detailed description that is given below with reference to the appended drawings as follows:

FIG. 1 is a perspective view of an autoinjector according to an embodiment of the invention,

FIGS. 2A and 2B are perspective views of a cap of an autoinjector according to an embodiment of the invention,

FIG. 3 is a bottom view of an autoinjector according to an embodiment of the invention,

FIG. 4A is a cross-section view of an autoinjector according to an embodiment of the invention, in the A-A plane of the FIG. 3,

FIG. 4B is a cross-section view of an autoinjector according to an embodiment of the invention, in the B-B plane of the FIG. 3,

FIG. 5A is a cross-section view of an autoinjector according to an embodiment of the invention, in the A-A plane of the FIG. 3, when the cap is removed from the housing,

FIG. 5B is a cross-section view of an autoinjector according to an embodiment of the invention, in the B-B plane of the FIG. 3, when the cap is removed from the housing,

FIG. 6A is a cross-section view of an autoinjector according to an embodiment of the invention, without the cap,

FIG. 6B is a cross-section of the autoinjector of FIG. 6A, the cap being attached to the housing,

FIGS. 7A, 7B, 7C are cross-section views of an autoinjector according to an embodiment of the invention, the needle cover being, respectively, in the first extended position (ready-to-use position), the retracted position, and the second extended position (safety position),

FIG. 8 is a perspective cross-section view of a retainer of an autoinjector according to an embodiment of the invention,

FIG. 9A is a perspective view of an autoinjector according to an embodiment of the invention, the cap being removed from the housing,

FIG. 9B is a partial cross-section and perspective view of a cap of an autoinjector according to an embodiment of the invention.

### DESCRIPTION OF THE INVENTION

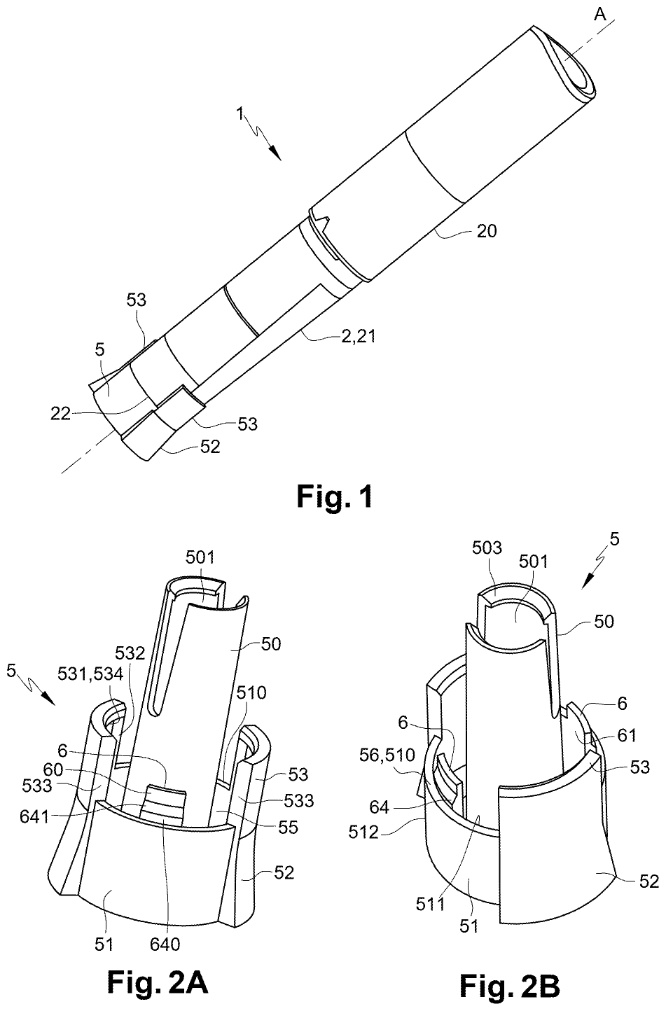

With reference to FIG. 1 is shown an autoinjector 1 according to an embodiment of the invention. The autoinjector 1 is designed for automatic injection of a product into an injection site. The autoinjector 1 extends along a longitudinal axis A. The autoinjector 1 includes a housing 2 comprising a top body 20 and a bottom body 21 assembled to each other by any appropriate securing means such as, for instance, snap-fitting means, a cap 5 removably attached to the housing 2, and a needle cover 4 axially movable within the housing 2.

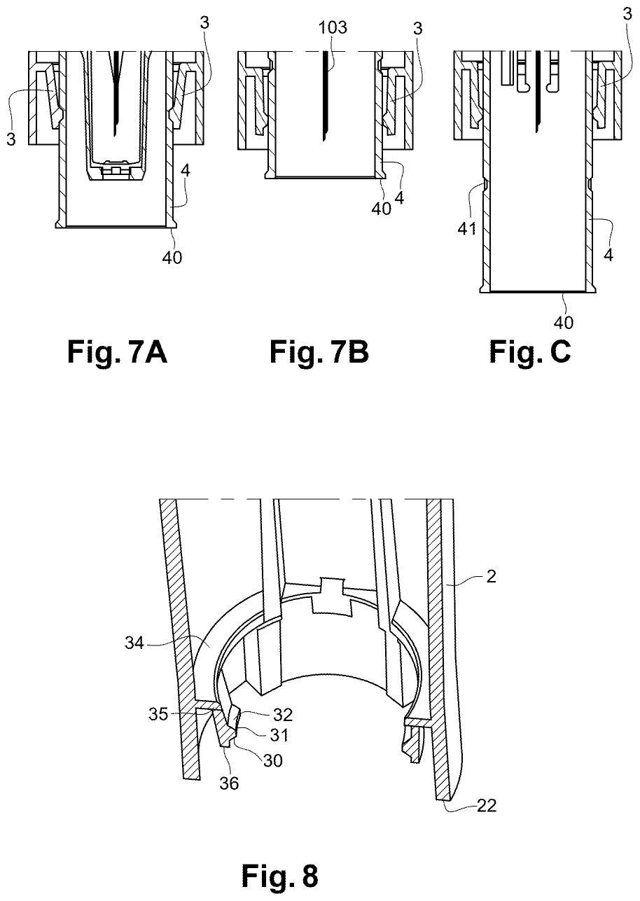

The needle cover 4 is arranged within the bottom body 21 and a safety spring 45 (not shown) for urging the needle cover 4 in a distal direction. With reference to FIGS. 7A, 7B, 7C, the needle cover 4 is axially movable along the longitudinal axis A with respect to the bottom body 21 between a first extended position (ready-to-use position, FIG. 7A), in which the needle cover 4 at least partially or completely shields the injection needle 103, a retracted position (activated position, FIG. 7B), proximally located relative to said first extended position, in which the needle cover 4 moves inside the autoinjector 1 to activate the injection mechanism and thus trigger the injection operation (in which the injection spring moves the plunger rod in the distal direction, thus pushing the stopper 105 to expel the medical product contained within the barrel 101), and a second extended position (safety position, FIG. 7C) in which after injection the needle cover 4 moves back in the distal direction to safely shield the injection needle 103.

The needle cover 4 has a distal end designed to be pressed against an injection site. Movement of the needle cover 4 from the first extended position (FIG. 7A) to the retracted position (FIG. 7B) is thus caused by the user pressing the distal end 40 of the needle cover 4 against the injection site, and movement of the needle cover 4 from the retracted position (FIG. 7B) to the safety position (FIG. 7C) is due to the safety spring 45 pushing the needle cover 4 back in the distal direction.

With reference to FIG. 4A, the needle cover 4 further includes a retaining feature, such as a radial recess 41, arranged on an outer lateral surface 42 of the needle cover 4 for retaining the needle cover 4 in the first extended position, as will be explained in further details below. The radial recess 41 defines a proximal abutment surface 43, and may further define an opposite distal abutment surface 44.

The housing 2 (more specifically the bottom body 21) is configured to receive a medical container 100 such as a syringe, for instance a prefilled syringe. The medical container 100 has a tubular barrel 101 defining a reservoir for containing a medical product to be injected. The barrel 101 has a distal end 102 provided with an injection needle 103 and a needle shield removably attached to said distal end for protecting and sealing the injection needle 103. The barrel 101 has an opened proximal end (not shown) which may be provided with a flange and which is configured to receive a plunger rod urged in the distal direction by an injection spring. The plunger rod is configured to push a stopper 105 arranged inside the barrel 101, thereby expelling the medical product. The top body 20 of the housing 2 receives an injection mechanism which includes the plunger rod, the injection spring and any other appropriate means known from the art for triggering and performing an injection operation.

The housing 2 (more specifically the bottom body 21) has an opened distal end, as shown for instance in FIG. 4A, and a retainer 3 for retaining the needle cover 4 in the first extended position. The retainer 3 extends from an inner lateral wall 23 of the housing 2 for engaging the retaining feature, i.e. the radial recess 41, of the needle cover 4. The retainer 3 is preferably arranged at the distal end 22 of the housing 2 to cooperate with a locking tab 6 of the cap 5, as will be explained below.

The retainer 3 includes a distal abutment surface configured for abutting against the proximal abutment surface 43 of the needle cover 4. The abutment of the distal abutment surface 30 of the retainer 3 against the proximal abutment surface 43 of the needle cover 4 prevents the needle cover 4 from moving to the retracted position. That is, the distal abutment surface 30 of the retainer 3 blocks a proximal movement of the needle cover 4. The distal abutment surface 30 of the retainer 3 and the proximal abutment surface 43 of the needle cover 4 may be inclined with regard to the central longitudinal axis A. Therefore, the retainer 3 allows for movement of the needle cover 4 in the proximal direction when the proximal force exerted on a distal end of said needle cover 4 exceeds a predetermined threshold, i.e. when the distal end 40 of the needle cover 4 is pressed by a user against the injection site to perform the injection operation, but blocks a proximal movement should the needle cover 4 be accidentally urged in the proximal direction, i.e. for instance if the autoinjector 1 falls down on the floor cap 5 upward. The distal abutment surface may be arranged at a distal side of an inward radial protrusion 31. The protrusion of the retainer 3 and the radial recess 41 of the needle cover 4 may be complementarily shaped. The proximal abutment surface 32 of the retainer 3 may be formed at a proximal side of the protrusion.

The retainer 3 may further include a proximal abutment surface, which may be arranged at a proximal side of the protrusion, for abutting against the distal abutment surface 44 of the needle cover 4 in order to lock the needle cover 4 in the first extended position.

The retainer 3 has an outer lateral surface 33 arranged opposite said protrusion for receiving a locking tab 6 of the cap 5. The locking tab 6 thus exerts a radial inward force against said outer lateral surface 33 for keeping the retainer 3 of the housing 2 engaged with the needle cover 4.

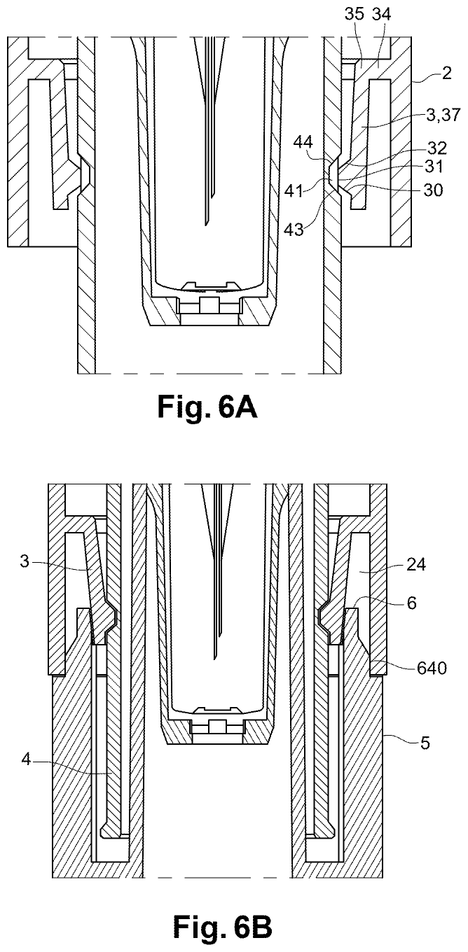

The retainer 3 is outwardly radially movable with respect to the needle cover 4, between an axial blocking position (FIG. 4A), in which the distal abutment surface 30 of the retainer 3 axially abuts against the proximal abutment surface 43 of the needle cover 4, and a release position (FIG. 6A), in which the distal abutment surface 30 of the retainer 3 is shifted away from the axial path of the proximal abutment of the needle cover 4 such that the needle cover 4 moves towards the retracted position due to the pressure exerted by its distal end on the injection site.

The retainer 3 may be in the form of a flexible leg 37 radially deformable between the blocking position and the release position. In the embodiment illustrated in FIGS. 4A-4B, the blocking position is a rest (non deformed) position of the retainer 3, while the release position is a deformed position of the retainer 3.

The retainer 3 may include a circumferential rib 34 for connecting the (fixed end 35 of the) flexible leg 37 to the inner lateral wall 23 of the housing 2. The circumferential rib 34 also helps delimit an accommodation room 24 for receiving the locking tab 6 of the cap 5 without risk of blockage of the locking tab 6.

The flexible leg 37 of the retainer 3 has a fixed end 35 secured to the housing 2, and an opposite free end which may include the protrusion. The free end includes, on an inner side, the distal abutment of the retainer 3, and on an outer side, the outer lateral surface 33 that receives the locking tab 6. Preferably, the free end is distally located with respect to the fixed end 35. Thus, the retainer 3 acts as a ratchet that prevents movement of the needle cover 4 in the proximal direction. The retainer 3 may be inclined such that, in the blocking position, the free end is closer to the central longitudinal axis A than the fixed end 35. As a result, the accommodation room 24 defined between the retainer 3 and the housing 2 widens in the distal direction. This allows reliable insertion of the locking tab 6 in the accommodation when the cap 5 is attached to the housing 2.

Preferably, the free end 36 of the retainer 3 is proximally located with respect to the distal end 22 of the housing 2. That is, the retainer 3 is fully contained within the housing 2 and does not distally extend outside the housing 2, beyond the distal end of said housing 2.

The retainer 3 is secured to the housing 2 by means of the fixed end 35 and, if need be, of the circumferential rib 34. That is, the retainer 3 is integral with the housing 2. Preferably, the retainer 3 and the housing 2 are made of a single piece.

The cap 5 of the autoinjector 1 is removably attached to the housing 2 in order to close the opened distal end 22 of the bottom body 21. The cap 5 includes a remover 50 for removing the needle shield 104, an external wall 51 extending around the remover 50 for protecting the remover 50 and for securing the cap 5 to the housing 2, and a connecting member 54 for rigidly connecting the remover 50 to the external wall 51.

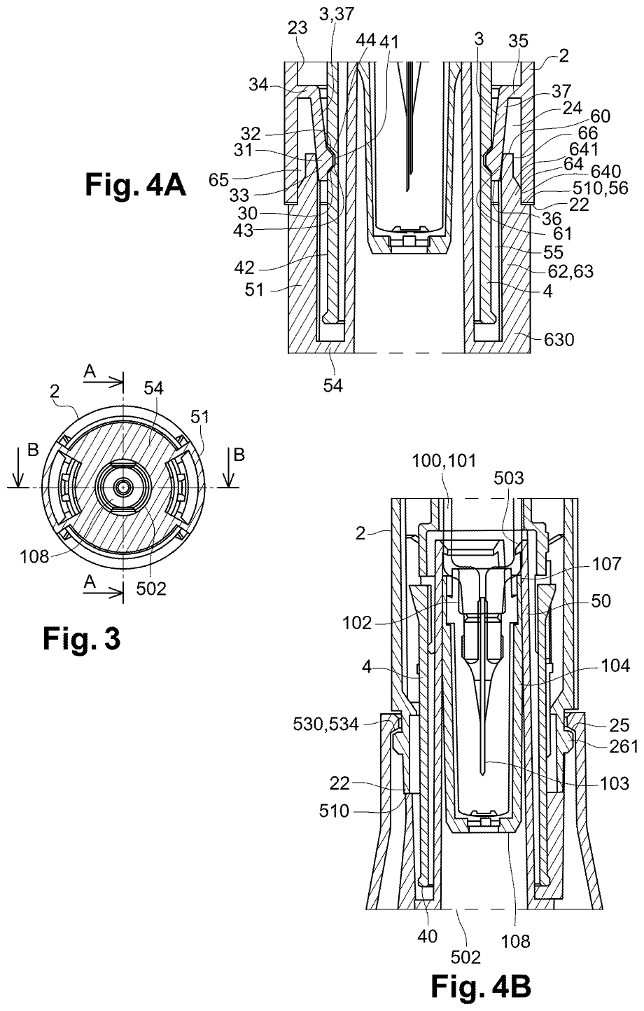

The remover 50 is arranged within the external wall 51 for engaging the needle shield 104 when the cap 5 is attached to the housing 2, and is configured for removing the needle shield 104 when the cap 5 is removed from the housing 2. The remover 50 may be in the form of an axial sleeve having an opened proximal end 501 for receiving the needle shield 104, and an opposite opened distal end 502, FIG. 3, giving access to a distal end 108 of the needle shield 104 for performing a push-back step of the medical container 100 during assembly of the autoinjector 1. The remover 50 includes a hook 503 or any appropriate means for engaging a proximal end 107 of the needle shield 104 such that removal of the cap 5 from the housing 2 by the user entails removal of the needle shield 104 from the medical container 100, thereby uncovering the injection needle 103 before injection.

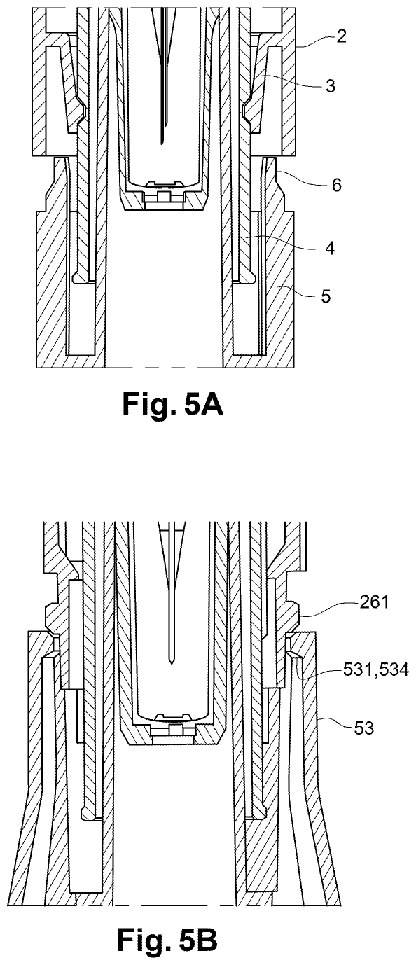

The remover 50 is connected to the external wall 51 by means of the connecting member 54, which may arranged at the distal end of the cap 5. The connecting member 54 may be a disk or end-plate which is in the form of a transversal wall orthogonal to the longitudinal axis A. The connecting member 54 more specifically connects the distal end of the remover 50 to the distal end of the external wall 51. The connecting member 54 provides a distance between the remover 50 and the external wall 51, such that the remover 50 and the external wall 51 delimit a circumferential cavity 55 for accommodating the needle cover 4, FIG. 5A.

The external wall 51 may include grasping means, for instance in the form of two diametrically opposite outer grasping surfaces 52 which are inclined relative to the longitudinal axis A for easing withdrawal of the cap 5. As illustrated in FIGS. 2A-2B, the grasping surfaces 52 may taper in a proximal direction.

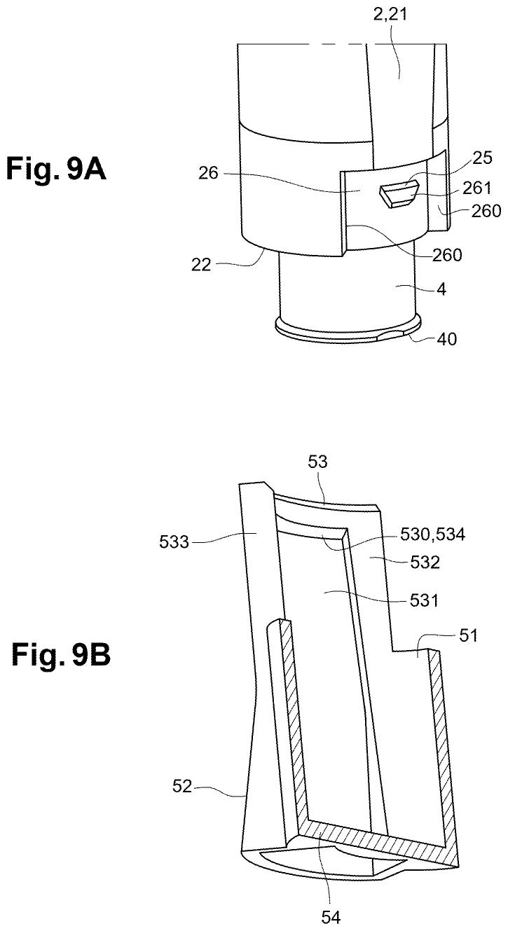

The external wall 51 further includes securing means for removably securing the cap 5 to the housing 2. The securing means may be configured for securing the cap 5 in both the axial and the rotational directions. In the embodiment illustrated in FIGS. 2A-2B, the securing means include two securing legs 53 proximally protruding from a distal edge 510 of the external wall 51. The distal edge 510 of the external wall 51 is configured for abutting against the distal end 22 of the housing 2 to stop the cap 5 in the proximal direction. The two diametrically opposite securing legs 53 preferably extend in the continuity of the grasping surfaces 52, i.e. they run alongside the grasping surfaces 52. The securing legs 53 include a distally oriented stop 530, which may be arranged at a proximal end of the securing legs 53 for abutting against a proximally oriented stop 25 of the housing 2, FIG. 9A. The distally oriented stop 530 may be a distal shoulder 534 defined by a radial recess 531 extended through an inner lateral surface 532 of the securing legs 53, FIG. 9B. The housing 2 may include an external recess 26 for receiving the securing legs 53. The external recess 26 of the housing 2 and the securing legs 53 may be complementarily shaped. The proximally oriented stop 25 of the housing 2 may be arranged within the external recess 26 for engaging the complementary shaped distally oriented stop 530 of the securing legs 53. The proximal stop may be in the form of a radial protrusion 261 which may taper in the distal direction to favor attachment of the cap 5 to the housing 2.

Still with reference to FIGS. 2, 9A and 9B, the securing legs 53 define an orthoradial edge 533 for abutting against a corresponding orthoradial shoulder 260 of the external recess 26 of the housing 2. This prevents any rotation of the cap 5 when the cap 5 is attached to the housing 2. The orthoradial edge 533s and shoulders also provide indication of the appropriate rotational position of the cap 5 relative to the housing 2 that allows for engagement of a locking tab 6 of the cap 5 with the retainer 3 of the housing 2 when the cap 5 is being mounted to the housing 2.

The external wall 51 of the cap 5 includes a locking tab 6 which proximally extends above the distal edge 510 of the external wall 51 for locking the retainer 3 of the housing 2 in the blocking position. The locking tab 6 may be in the form of two diametrically opposite locking tabs 6 which may be arranged at 90° from the securing legs 53. The locking tabs 6 include a supporting portion 62 for securing the locking tabs 6 to the cap 5, and a locking portion for locking the retainer 3 in the axial blocking position.

The supporting portion 62 may include an axial rib 63 secured to an inner lateral surface 511 of the external wall 51 and radially inwardly protruding from said inner lateral surface 511, and/or a stiffener 64 secured to the distal edge 510 of the external wall 51, and/or a distal end 630 secured to a proximal side of the connecting member 54. The stiffener 64 includes an outer abutment surface 640 for abutting against the inner lateral wall 23 of the housing 2. The stiffener 64 may also include a chamfer 641 or any other inclined surface that may abut against the distal end 22 of the housing 2 for easing insertion of the locking tabs 6 into the accommodation room 24s when the cap 5 is being mounted onto the housing 2. The stiffener 64 proximally protrudes from the distal edge 510 of the external wall 51, but may however remains distant from an outer lateral surface 512 of the external wall 51 so that a proximal shoulder 56 is defined between the stiffener 64 and the outer lateral surface 512 to abut against the distal end 22 of the housing 2.

The locking portion may be arranged at a free proximal end 60 of the locking tabs 6 for engaging inside the accommodation room 24 and abutting against the outer lateral surface 33 of the retainer 3, thereby locking the retainer 3 in the axial blocking position. To that end, the locking portion includes an inner side surface 61 that abuts against the outer lateral surface 33 of the retainer 3. The locking portion may be distant from the inner lateral wall 23 of the housing 2 and may thus delimit a radial gap 65 between the housing 2 and the locking tab 6.

It is contemplated that the remover 50, the external wall 51, the connecting member 54 and the locking tabs 6 of the cap 5 may be integral with each other. In the embodiment illustrated for instance in FIGS. 2A, 2B or 4A, the remover 50, the external wall 51, the connecting member 54 and the locking tabs 6 may be made of a single piece.

The operation of the autoinjector 1 is described below.

In FIG. 4A, the cap 5 is attached to the housing 2. The retainer 3 is in the blocking position: the retainer 3 engages the needle cover 4 and axially blocks the needle cover 4 in the pre-use position. If the autoinjector 1 falls down on the floor, the needle cover 4 is thus prevented from moving to the retracted position and the autoinjector 1 is thus prevented from being inadvertently activated. The locking tabs 6 of the cap 5 abut against the retainer 3, thereby keeping the retainer 3 in the blocking position as long as the cap 5 is attached to the housing 2.

To perform an injection operation, the user has to grasp the cap 5 and pull in the distal direction to remove the cap 5 from the housing 2, FIG. 9A. Since the remover 50 of the cap 5 is engaged with the needle shield 104, removal of the cap 5 entails removal of the needle shield 104. Besides, the locking tabs 6 are removed together with the cap 5, so that the retainer 3 is able to move to the release position. Since the blocking position here is a rest position of the retainer 3, then the retainer 3 remains engaged with the needle cover 4 (FIGS. 5A, 7A).

The user presses the distal end 40 of the needle cover 4 against the injection site. The force exerted by the user causes the needle cover 4 to outwardly radially deflect the retainer 3. The retainer 3 moves to the release position and disengages the needle cover 4. The needle cover 4 moves to the retracted position (FIG. 7B) and triggers the injection mechanism.

When the injection is completed, the user withdraws the autoinjector 1 from the injection site. Due to the safety spring 45, the needle cover 4 moves back in the distal direction and passes over the retainer 3 until the needle cover 4 reaches the safety position (FIG. 7C).

FIGS. 6A-6B illustrate another embodiment wherein the blocking position of the retainer 3 corresponds to a deformed position of the retainer 3, while the release position corresponds to a rest (not deformed) position of the retainer 3. That is, the retainer 3 naturally remains in the release position as visible in FIG. 6A. In order to lock the needle cover 4, the retainer 3 must be deformed to the blocking position by means of the locking tabs 6 of the cap 5 which exert a radial inward force on the retainer 3 (FIG. 6B). As long as the cap 5 is attached to the housing 2, the locking tabs 6 maintain the retainer 3 engaged with the needle cover 4 in the axial blocking position.

The operation of the autoinjector 1 according to the embodiment of FIGS. 6A-6B is similar to the embodiment of FIGS. 4A-4B, except that the blocking position of the retainer 3 being the deformed position of said retainer 3, the flexible legs 37 of the retainer 3 automatically flex back to their rest position when the cap 5 is removed from the housing 2. Then, the retainer 3 does not interfere any longer with the needle cover 4.

It is readily understandable from the above description that the autoinjector 1 of the invention permits to prevent inadvertent activation of the injection mechanism during a drop test as long as the cap 5 is attached to the housing 2.

It is to be understood that the present invention is not limited to the embodiments described above and illustrated in the drawings; rather, the skilled person will recognize that many changes and modifications may be made within the scope of the appended claims.

1. An autoinjector, for automatic injection of a product into an injection site, said autoinjector comprising:
a housing extending along longitudinal axis A and having an opened distal end, said housing being configured to receive a medical container having a barrel defining a reservoir for containing a medical product, said barrel having a distal end provided with an injection needle,
a needle cover coupled to and axially movable with respect to said housing between a first extended position, in which the needle cover at least partially shields the injection needle, a retracted position, in which the needle cover moves proximally with respect to the housing to trigger injection of the medical product into an injection site, and a second extended position in which the needle cover moves back in the distal direction to shield the injection needle,
a cap removably attached to the opened distal end of the housing,
a retainer coupled to the housing for blocking a proximal movement of the needle cover from the first extended position to the retracted position as long as the cap is attached to the housing, wherein the retainer includes
a distal abutment surface which is radially movable between a blocking position in which said distal abutment surface abuts against a proximal abutment surface of the needle cover, and a release position in which said distal abutment surface is radially shifted away from the proximal abutment surface of the needle cover to allow movement of the needle cover in the proximal direction, and wherein the cap includes
a locking tab radially abutting against the retainer for maintaining the distal abutment surface of the retainer in the blocking position.

2. The autoinjector according to claim 1, wherein the locking tab has an outer abutment surface abutting against an inner lateral wall of the housing.

3. The autoinjector according to claim 1, wherein the retainer defines an accommodation room for receiving a free proximal end of the locking tab between an outer lateral surface of the retainer and an inner lateral wall of the housing.

4. The autoinjector according to claim 3, wherein an outer lateral surface of the free proximal end of the locking tab is separated from an inner lateral wall of the housing by a radial gap.

5. The autoinjector according to claim 3, wherein the locking tab has an axial rib distally extending from the free proximal end of the locking tab and said axial rib is fixed to an external wall of the retainer.

6. The autoinjector according to claim 5, wherein the axial rib has a distal end which is fixed to a connecting member that connects a cap external wall to a remover of the cap.

7. The autoinjector according to claim 3, wherein the locking tab has a stiffener arranged at an outer side of the free proximal end, the stiffener proximally protruding from a distal edge of the cap.

8. The autoinjector according to claim 7, wherein the stiffener includes an inclined surface for easing insertion of the locking tab inside the housing.

9. The autoinjector according to claim 1, wherein the retainer includes a flexible leg, said flexible leg having a fixed end secured to the housing and a free end defining the distal abutment surface of the retainer.

10. The autoinjector according to claim 9, wherein the free end of the flexible leg is distal to the fixed end.

11. The autoinjector according to claim 9, wherein the fixed end of the flexible leg includes an annular rib radially protruding from an inner lateral wall of the housing.

12. The autoinjector according to claim 9, wherein the blocking position of the retainer is a rest position of the flexible leg.

13. The autoinjector according to claim 9, wherein the release position of the retainer is a rest position.

14. The autoinjector according to claim 1, wherein the retainer has a proximal abutment surface abutting against a distal abutment surface of the needle cover for blocking the needle cover in the distal direction.

15. The autoinjector according to claim 1, wherein the autoinjector includes securing means for removably securing the cap to the housing in both axial and rotational directions.
